(19) United States
(12) Patent Application Publication
(10) Pub. No.: US 2012/0322906 A1
(43) Pub. Date: Dec. 20, 2012

(54) POLYMER AND METHODS OF PREPARING AND USING A POLYMER

(75) Inventors: Johan Billing, Lund (SE); Jonas Eriksson, Kavlinge (SE); Ola Karlsson, Lund (SE); Yan Liu, Hassleholm (SE); Christian Svensson Stark, Vaxjo (SE); Ecevit Yilmaz, Bjarred (SE)

(73) Assignee: Cargill, Incorporated, Wayzata, MN (US)

(21) Appl. No.: 13/519,160

(22) PCT Filed: Dec. 30, 2010
(86) PCT No.: PCT/US10/62578

§ 371 Date: Jun. 26, 2012

(51) Int. Cl. C08F 212/36; C07D 209/20

(52) U.S. Cl. 521/146; 548/495

(57) ABSTRACT

Methods for preparing a polymer using Hansen solubility parameters are described. Also described is a polymer having certain properties, including those related to the Hansen solubility parameters, having utility in the separation of monatin from a monatin-containing mixture.

### CROSS-REFERENCE TO RELATED APPLICATIONS

This application claims priority to U.S. Provisional Application Ser. No. 61/335,033, filed 30 Dec. 2009, entitled A POLYMER AND METHODS OF PREPARING AND USING A POLYMER, which is incorporated herein by reference in its entirety.

### FIELD

The present disclosure relates generally to a polymer and a method of preparing and using a polymer. The present disclosure also relates generally to a method and system for producing monatin. In particular, the present disclosure relates to a polymer and method of preparing and using a polymer to recover monatin from a monatin-containing mixture.

### BACKGROUND

Monatin (2-hydroxy-2-(indol-3-ylmethyl)-4-aminoglutaric acid) is a naturally occurring, high intensity or high potency sweetener that was originally isolated from the plant Sclerochiton ilicifolius, found in the Transvaal Region of South Africa. Monatin has the chemical structure:

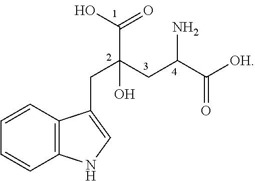

Because of various naming conventions, monatin is also known by a number of alternative chemical names, including: 2-hydroxy-2-(indol-3-ylmethyl)-4-aminoglutaric acid; 4-amino-2-hydroxy-2-(1H-indol-3-ylmethyl)-pentanedioic acid; 4-hydroxy-4-(3-indolylmethyl)glutamic acid; and 3-(1-amino-1,3-dicarboxy-3-hydroxy-but-4-yl)indole.

Monatin has two chiral centers thus leading to four potential stereoisomeric configurations: the R,R configuration (the “R,R stereoisomer” or “R,R monatin”); the S,S configuration (the “S,S stereoisomer” or “S,S monatin”); the R,S configuration (the “R,S stereoisomer” or “R,S monatin”); and the S,R configuration (the “S,R stereoisomer” or “S,R monatin”).

Reference is made to WO 2003/091396 A2, which discloses, inter alia, polypeptides, pathways, and microorganisms for in vivo and in vitro production of monatin. WO 2003/091396 A2 (see, e.g., FIGS. 1-3 and 11-13) and U.S. Patent Publication No. 2005/282260 describe the production of monatin from tryptophan through multi-step pathways involving biological conversions with polypeptides (proteins) or enzymes. One pathway described involves converting tryptophan to indole-3-pyruvate (“I3P”) (reaction (1)), converting indole-3-pyruvate to 2-hydroxy 2-(indol-3-ylmethyl)-4-keto glutaric acid (monatin precursor, “MP”) (reaction (2)), and converting MP to monatin (reaction (3)). The three reactions can be performed biologically, for example, with enzymes.

### SUMMARY

One embodiment is directed to a method of preparing a polymer in the presence of a solvent system, where the solvent system is selected such that it has a dispersion solubility parameter between about 15.9 and 18.3 MPa^{1/2}, a polar solubility parameter between about 4.0 and 6.2 MPa^{1/2} and a hydrogen bonding solubility parameter between about 5.5 and 12.7 MPa^{1/2}. In a further aspect, the solvent system is selected such that the polymer and the solvent system have a Skaarup distance (Ra) between the polymer and the solvent system of from about 7.7 MPa^{1/2} to 10.9 MPa^{1/2}. In another aspect is a polymer produced by the method.

A second embodiment is directed to a method of preparing a polymer in the presence of a solvent system, the solvent system being selected such that the polymer has an average pore diameter between about 50 Angstroms to 450 Angstroms. In an additional aspect is a polymer produced by the method.

Another embodiment is directed to a polymer adapted to recover monatin from a mixture, where the polymer has an average pore diameter between about 50 Angstroms to 450 Angstroms.

A further embodiment is directed to a method of recovering monatin from a mixture, where the method includes the step of using a polymer made in the presence of a solvent system selected such that it has a dispersion solubility parameter between about 15.9 and 18.3 MPa^{1/2}, a polar solubility parameter between about 4.0 and 6.2 MPa^{1/2} and a hydrogen bonding solubility parameter between about 5.5 and 12.7 MPa^{1/2}. In another aspect, the solvent system is selected such that the polymer and the solvent system have a Skaarup distance (Ra) between the polymer and the solvent system of from about 7.7 MPa^{1/2} to 10.9 MPa^{1/2}. The details of one or more non-limiting embodiments of the invention are set forth in the description below. Other embodiments of the invention should be apparent to those of ordinary skill in the art after consideration of the present disclosure.

### BRIEF DESCRIPTION OF THE DRAWINGS

FIG. 1 is a block diagram of an exemplary system for the separation and purification of monatin from a mixture including monatin, starting materials and intermediates.

FIG. 2 is a Loading Plot prepared using the software package SIMCA (Umetrics AB) showing the relationship between the original variables and the principle components for various polymers where the horizontal axis represents variables generally related to elution volume and the vertical axis represents variables generally related to resolution and where:

N=Plate Number for Sodium Nitrite

Average pore diameter (Å)=Average pore diameter assuming cylindrical pores

Peak pore diameter desorption (Å)=Dominant pore size in dV/d(log d) desorption plot

Swelling=Swelling index in ethanol (V_{wet}/V_{dry})

Density=Powder density of dry polymer

BV M=Elution volume for M in bed volumes (calc. from Rt)

BV MP=Elution volume for MP in bed volumes (calculated)

Surface area (m2/g)=Surface area of the polymer

Pore volume (m1/g)=Pore volume of the polymer

Rs M-MP=Resolution between monatin and monatin precursor

FIG. 3 is a Score Plot prepared using the software package SIMCA (Umetrics AM) showing the distribution of various polymers according to the Skaarup distance to polystyrene.

FIG. 4 is a Score Plot prepared using the software package SIMCA (Umetrics AM) showing the distribution of various polymers according to the Skaarup distance to polystyrene showing common relationships between various batches.

FIG. 5 is a scanning electron microscopy picture of Resin Batch #1 showing both the surface and the interior of polymer bead.

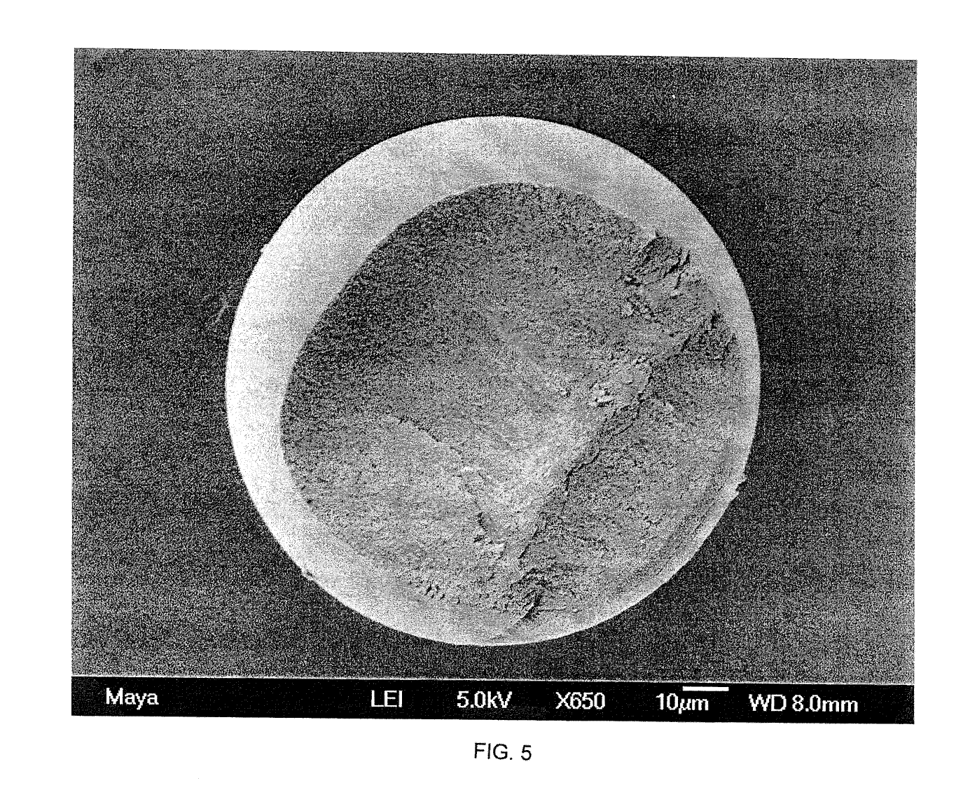

### DETAILED DESCRIPTION

The present disclosure is directed to a polymer (also referred to interchangeably herein as a resin and copolymer) and a method of preparing and using the polymer. In one embodiment, the polymer is adapted to recover monatin from a mixture including monatin where the mixture may include starting materials used in the production of monatin, and intermediates formed during the production of monatin. The recovered monatin has a purity of at least 90%. In some embodiments, the recovered monatin has a purity of at least 95%. In some embodiments, the recovered monatin is a sterioisomerically enriched R,R monatin. In some embodiments the polymer is a reverse phase resin. In some embodiments, the reverse phase resin is formed from a polystyrene/divinylbenzene copolymer. The polymer may be packed in a chromatography unit such as a dynamic axial compression (DAC) column.

Monatin has an excellent sweetness quality, and depending on a particular composition, monatin may be several hundred times sweeter than sucrose, and in some cases thousands of times sweeter than sucrose. As stated above, monatin has four stereoisomeric configurations. The S,S stereoisomer of monatin is about 50-200 times sweeter than sucrose by weight. The R,R stereoisomer of monatin is about 2000-2400 times sweeter than sucrose by weight. As used herein, unless otherwise indicated, the term “monatin” is used to refer to compositions including any combination of the four stereoisomers of monatin (or any of the salts thereof), including a single isomeric form.

Monatin may be synthesized in whole or in part by one or more of a biosynthetic pathway, chemically synthesized, or isolated from a natural source. If a biosynthetic pathway is used, it may be carried out in vitro or in vivo and may include one or more reactions such as the equilibrium reactions provided below as reactions (1)-(3). In one embodiment, is a biosynthetic production of monatin via enzymatic conversions starting from tryptophan and pyruvate and following the three equilibrium reactions below:

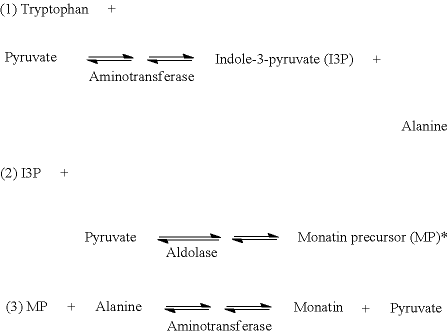

* Monatin precursor (MP) is 2-hydroxy 2-(indol-3-ylmethyl)-4-keto glutaric acid.

The following side-reactions may also occur, resulting in production of hydroxymethyl-oxo-glutarate (HMO), hydroxymethylglutamate (HMG) or a combination thereof:

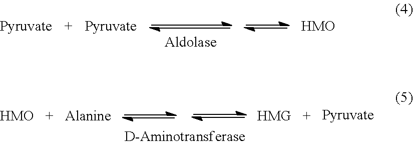

In the pathway shown above, in reaction (1), tryptophan and pyruvate are enzymatically converted to indole-3-pyruvate (I3P) and alanine in a reversible reaction. As exemplified above, an enzyme, here an aminotransferase, is used to facilitate (catalyze) this reaction. In reaction (1), tryptophan donates its amino group to pyruvate and becomes I3P. In reaction (1), the amino group acceptor is pyruvate, which then becomes alanine as a result of the action of the aminotransferase. The amino group acceptor for reaction (1) is pyruvate; the amino group donor for reaction (3) is alanine. The formation of indole-3-pyruvate in reaction (1) can also be performed by an enzyme that utilizes other α-keto acids as amino group acceptors, such as oxaloacetic acid and α-keto-glutaric acid. Similarly, the formation of monatin from MP (reaction (3)) can be performed by an enzyme that utilizes amino acids other than alanine as the amino group donor. These include, but are not limited to, aspartic acid, glutamic acid, and tryptophan.

Some of the enzymes useful in connection with reaction (1) may also be useful in connection with reaction (3). For example, aminotransferase may be useful for both reactions (1) and (3). The equilibrium for reaction (2), the aldolase-mediated reaction of indole-3-pyruvate to form MP (i.e. the aldolase reaction), favors the cleavage reaction generating indole-3-pyruvate and pyruvate rather than the addition reaction that produces the alpha-keto acid precursor to monatin (i.e. MP). The equilibrium constants of the aminotransferase-mediated reactions of tryptophan to form indole-3-pyruvate (reaction (1)) and of MP to form monatin (reaction (3)) are each thought to be approximately one. Methods may be used to drive reaction (3) from left to right and prevent or minimize the reverse reaction. For example, an increased concentration of alanine in the reaction mixture may help drive forward reaction (3). Reference is made to US Publication No. 2009/0198072 (application Ser. No. 12/315,685), which is also assigned to Cargill, the assignee of this application.

The overall production of monatin from tryptophan and pyruvate is referred to herein as a multi-step pathway or a multi-step equilibrium pathway. A multi-step pathway refers to a series of reactions that are linked to each other such that subsequent reactions utilize at least one product of an earlier reaction. In such a pathway, the substrate (for example, tryptophan) of the first reaction is converted into one or more products, and at least one of those products (for example, indole-3-pyruvate) can be utilized as a substrate for the second reaction. The three reactions above are equilibrium reactions such that the reactions are reversible. As used herein, a multi-step equilibrium pathway is a multi-step pathway in which at least one of the reactions in the pathway is an equilibrium or reversible reaction.

Because the R,R stereoisomer of monatin is the sweetest of the four stereoisomers, it may be preferable to selectively produce R,R monatin. For purposes of this disclosure, the focus is on the production of R,R monatin. However, it is recognized that the present disclosure is applicable to the production of any of the stereoisomeric forms of monatin (R,R; S,S; S,R; and R,S), alone or in combination.

In some embodiments, the monatin consists essentially of one stereoisomer—for example, consists essentially of S,S monatin or consists essentially of R,R monatin. In other embodiments, the monatin is predominately one stereoisomer—for example, predominately S,S monatin or predominately R,R monatin. “Predominantly” means that of the monatin stereoisomers present in the monatin, the monatin contains greater than 90% of a particular stereoisomer. In some embodiments, the monatin is substantially free of one stereoisomer—for example, substantially free of S,S monatin. “Substantially free” means that of the monatin stereoisomers present in the monatin, the monatin contains less than 2% of a particular stereoisomer. In some embodiments, the monatin is a stereoisomerically-enriched monatin mixture. “Stereoisomerically-enriched monatin mixture” means that the monatin contains more than one stereoisomer and at least 60% of the monatin stereoisomers in the mixture is a particular stereoisomer. In other embodiments, the monatin contains greater than 60%, 65%, 70%, 75%, 80%, 85%, 90%, 95%, 98% or 99% of a particular monatin stereoisomer. In another embodiment, a monatin composition comprises a stereoisomerically-enriched R,R-monatin, which means that the monatin comprises at least 60% R,R monatin. In other embodiments, stereoisomerically-enriched R,R-monatin comprises greater than 60%, 65%, 70%, 75%, 80%, 85%, 90%, 95%, 98% or 99% of R,R monatin.

For example, to produce R,R monatin using the three-step pathway shown above (reactions (1)-(3)), the starting material may be D-tryptophan, and the enzymes may be a D-aminotranferase and an R-specific aldolase. The three reactions, which are shown below, may be carried out in a single reactor or a multiple-reactor system.

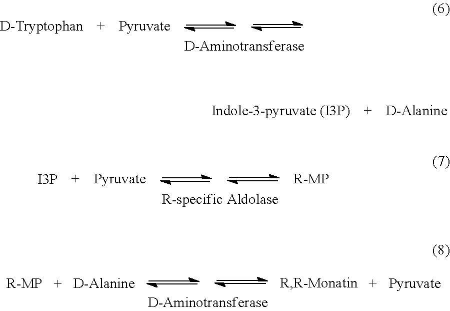

In an embodiment in which a single reactor is used, the two enzymes (i.e. the D-aminotransferase and the R-specific aldolase) may be added at the same time and the three reactions may run simultaneously. The same enzyme may be used to catalyze reactions (6) and (8). A D-aminotransferase is an enzyme with aminotransferase activity that selectively produces, in the reactions shown above, D-alanine and R,R-monatin. An R-specific aldolase is an enzyme with aldolase activity that selectively produces R-MP, as shown in reaction (7) above. Although a focus in the present disclosure is on R,R monatin, it is recognized that the method and system of separating and purifying monatin is applicable to any of the stereoisomeric forms of monatin.

There are multiple alternatives to the above pathway (i.e. reactions (6)-(8)) for producing R,R-monatin. For example, L-tryptophan may be used as a starting material instead of D-tryptophan. In that case, an L-aminotransferase may be used to produce indole-3-pyruvate and L-alanine from L-tryptophan. Because L-alanine is produced, this pathway may require the use of an alanine racemase to convert the L-alanine to D-alanine, thus adding a fourth reaction to the monatin production pathway. (D-alanine is required to produce R,R monatin from the R— stereoisomer of monatin precursor (R-MP). In addition to requiring another enzyme (alanine racemase), undesired side reactions may also occur in this pathway. For example, L-alanine may react with the L-aminotransferase to produce R,S-monatin, or D-alanine may react with I3P to form D-tryptophan, resulting in a racemate of L-tryptophan and D-tryptophan, which has poor solubility. Some disadvantages of this pathway may be avoided by using a two reactor system as opposed to a single reactor system. It is recognized that there are additional alternatives not specifically disclosed herein for performing the three-step equilibrium pathway to produce monatin. The method and system described herein for separating and purifying monatin is applicable to monatin produced using alternative pathways to what is disclosed herein.

As described above, in some pathways, it may be preferable to perform the monatin producing reactions in two or more separate reactors, while in other pathways it may be preferable to use a single reactor system. The decision to use a one reactor or a multiple reactor system may depend, in part, on whether D-tryptophan or L-tryptophan is used as a starting material. A single reactor system is obviously simpler in design, eliminating the need for a second reactor, as well as eliminating, in some cases, a need for a separation step between the first and second reactors. It is recognized that the method and system described herein for separating and purifying monatin may be used in combination with both a single reactor system and a multiple reactor system for the production of monatin.

Although the present disclosure focuses on the production of monatin using the biosynthetic multi-step equilibrium pathway described above, monatin may also be produced chemically or using a combination of both chemical synthesis and an enzymatic pathway. Regardless of the method used to produce monatin, the resulting monatin may be present in a mixture that contains other components, including starting materials, intermediates, side products of the monatin-producing reactions or combinations thereof. It is preferable to separate the monatin from these other components, which may include, for example, tryptophan, pyruvate, alanine, I3P, MP, HMG and HMO.

One purpose of the polymer described herein and application of such polymer is to recover as close to 100% as possible of the monatin produced at a high purity level. It is recognized that although it may be possible to recover essentially all of the monatin produced, if the monatin is not “pure” monatin, it is not defined herein as “recovered” monatin. As used herein, “pure” monatin is defined as a composition containing at least 90% by weight monatin, which is defined on a dry weight basis and corrected for inorganic counter ions. In some embodiments, the purity may be at least 90% in other embodiments, at least 95%. In some embodiments, is a method of recovering monatin from this mixture through chromatography for example, such that the monatin has at least 90% purity. In another embodiment, is a method of preparing a polymer adapted to recover monatin from this mixture through chromatography for example, such that the monatin has at least 95% purity. Although the present disclosure reports recovery of monatin from a monatin-containing mixture using the polymer described herein, the polymer and methods of making and using the polymer may be used with other mixtures to recover other materials of interest.

In one aspect, recovery is defined herein as the amount of pure monatin that is recovered from the mixture based on the starting mass of monatin. In some embodiments, about 80% by weight of the monatin, also on a dry weight basis, is recovered from the monatin-containing mixture. It is recognized that, in other embodiments, the system may be designed to recover less than 80% by weight of the monatin and/or recover monatin having a purity of less than 90%. It may be more efficient to recover less than 80%, depending, for example, on an overall system design for monatin production.

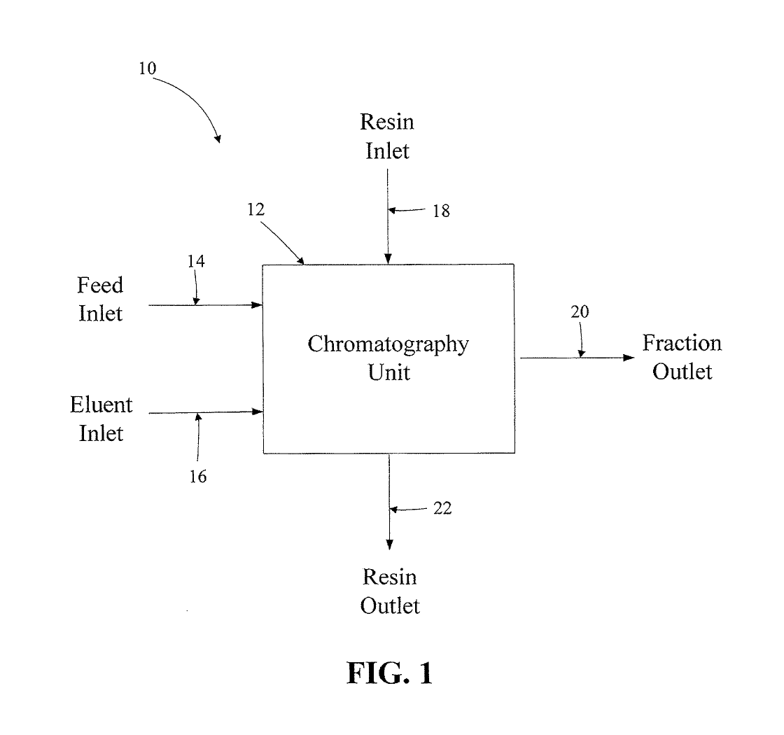

FIG. 1 is a block diagram of an exemplary system for the separation and purification of monatin using the polymer (also referred to as resin) of the present invention. System 10 includes chromatography unit 12, feed inlet 14, eluent inlet 16, resin inlet 18, fraction outlet 20 and resin outlet 22. In some embodiments, chromatography unit 12 is located downstream of an enzyme removal unit. Chromatography unit 12 is a column packed with resin that forms a stationary phase in the chromatography separation process. The column is packed by loading resin into the column through resin inlet 18. At the end of operation, the resin may be removed from the column through resin outlet 22. In some embodiments, the feed material (i.e. the monatin-containing mixture) is injected into the column through feed inlet 14. The monatin-containing mixture may include tryptophan, pyruvate, monatin, MP, I3P, alanine, HMO and HMG. The components in the monatin-containing mixture are adsorbed by the packed resin in the column. A mobile phase (an eluent) passes through the column through eluent inlet 16 and is designed to elute the components from the column through fraction outlet 20.

In some embodiments, a pump is used to inject the monatin-containing mixture into chromatography unit 12. The resin inside chromatography unit 12 causes the various components in the mixture to adsorb to the resin particles based on each component's affinity for the resin. The eluent is then pumped into chromatography unit 12 through eluent inlet 16. As the eluent passes through the column the components adsorbed by the resin in the column are eluted and flow out through the column with the eluent via fraction outlet 20. The most weakly adsorbed components (those with the lowest affinity for the resin) elute first. The most strongly adsorbed (i.e. highest affinity) elute last. The components may thus be separated by taking different fractions from the column. This may be done, for example, by transferring the outlet stream from fraction outlet 20 into a different container for each fraction.

In some embodiments, chromatography unit 12 uses reversed phase chromatography, meaning that the stationary phase or resin is non-polar. As compared to “normal” chromatography which uses a hydrophilic surface chemistry having a stronger affinity for polar compounds, in reversed phase chromatography the elution order of the components is reversed. The polar compounds are eluted first while non-polar compounds are retained. Thus the resin in reversed phase chromatography may be any inert non-polar substance; however, the particular composition of the resin may directly impact the separation behavior of the mixture, and small changes in the surface chemistries as well as the physical parameters of the resin may lead to important changes in selectivity. In one embodiment of the present invention, the resin used for reversed phase chromatography is a macroporous stryrene-divinylbenzene co-polymer. In one aspect, the divinylbenzene acts as a crosslinker and provides rigidity in the polymer beads and therefore also to the packed bed. In another aspect, the polymer includes from about 30 to 100% of divinylbenzene. In still another aspect, the polymer includes from about 60 to 100% of divinylbenzene. And in yet another aspect, the polymer includes from about 79 to 100% of divinylbenzene. The solvent (also referred to herein as a porogen) or solvent system used to prepare the resin may also impact the surface chemistries as well as the physical parameters of the resin. For example, and as shown by the data in Tables 1 and 2, recovery of monatin from a monatin-containing mixture is improved using smaller resin particles with the same or narrower particle size distribution. Table 1 compares the monatin separation performance for sieved fractions 70-90 μm of three different polystyrene-divinylbenzene resins. Resin batch #1 (MH07-1234) was formed in the presence of a benzyl alcohol and chloroform solvent system, where Resin Batch #1 is being shown as the control resin. Resin batches #17 (RH12-1517) and #20 (RI02-1602) were formed in the presence of a benzyl alcohol, toluene and methyl isobutyl ketone solvent system. Resin batch #17 was optimized on resolution and Resin batch #20 was optimized on decreasing the number of bed volumes of mobile phase for elution and swelling.

TABLE 1
Comparison of three different resins

| | | MP/Monatin | Monatin | Monatin/ | I3P Elution | | Swelling |
|---|---|---|---|---|---|---|---|
| Resin Batch # | Comment | Rs | Elution BV | I3P Rs | BV | Plates N | Index |
| 1 | MH07-1234 | 1.32 | 3.1 | 1.5 | 6.9 | 185 | 1.4 |
| 17 | RH12-1517 | 1.45 | 2.6 | 1.7 | 5.2 | 419 | 1.2 |
| | Improvement %* | 9 | 19 | 12 | 33 | | 17 |
| 20 | RHI02-1602 | 1.09 | 1.9 | 1.55 | 3.5 | 389 | 1.1 |
| | Improvement %* | −21 | 63 | 3 | 97 | | 27 |

*Improvement in percent performance after optimization compared to original reverse phase resin. Percentages calculated with Batch #1 as base level.

It can clearly be seen in Table 1 that Resin batch #20 is better on many of the parameters that are assessed; 63% faster elution of monatin, 97% faster elution of I3P and 27% lower degree of swelling. However, the peak resolution of MP and monatin for resin batch #3 is lower than batches #1 or #17. Resin batch #20 requires a lower number of bed volumes of mobile phase for elution and thus less water required to elute monatin, as compared to both batches #17 and #20.

A similar comparison is shown in Table 2, where Resin batch #1 with particle sizes of 70-90 μm were compared to Resin batches #25 (RI04-1675) and #24 (RI05-1744) formed in the presence of a benzyl alcohol, toluene and methyl isobutyl ketone solvent system and having particle sizes of 32-50 μm.

TABLE 2
Comparison of three different resins

| | | MP/Monatin | Monatin | Monatin/ | I3P Elution | | Swelling |
|---|---|---|---|---|---|---|---|
| Resin Batch # | Comment | Rs | Elution BV | I3P Rs | BV | Plates N | Index |
| 1 | MH07-1234 | 1.32 | 3.1 | 1.5 | 6.9 | 185 | 1.4 |
| 25 | RI04-1675 | 2.72 | 3.1 | 3.18 | 6.9 | 1197 | 1.2 |
| | Improvement %* | 51 | 0 | 53 | 0 | | 17 |
| 24 | RI05-1744 | 2.17 | 2.3 | 2.6 | 4.5 | 1015 | 1.1 |
| | Improvement %* | 39 | 39 | 42 | 53 | | 27 |

*Improvement in percent performance after optimization compared to Batch #1.

The values reported in Table 2 show that the improvement in resolution performance is significant with the smaller particle size but with a slight increase in the number of bed volumes needed for separations. Resin batch #24 is significantly better than Resin batch #1 in terms of faster elution of monatin (i.e. fewer bed volumes are required), faster elution of I3P and a lower degree of swelling.

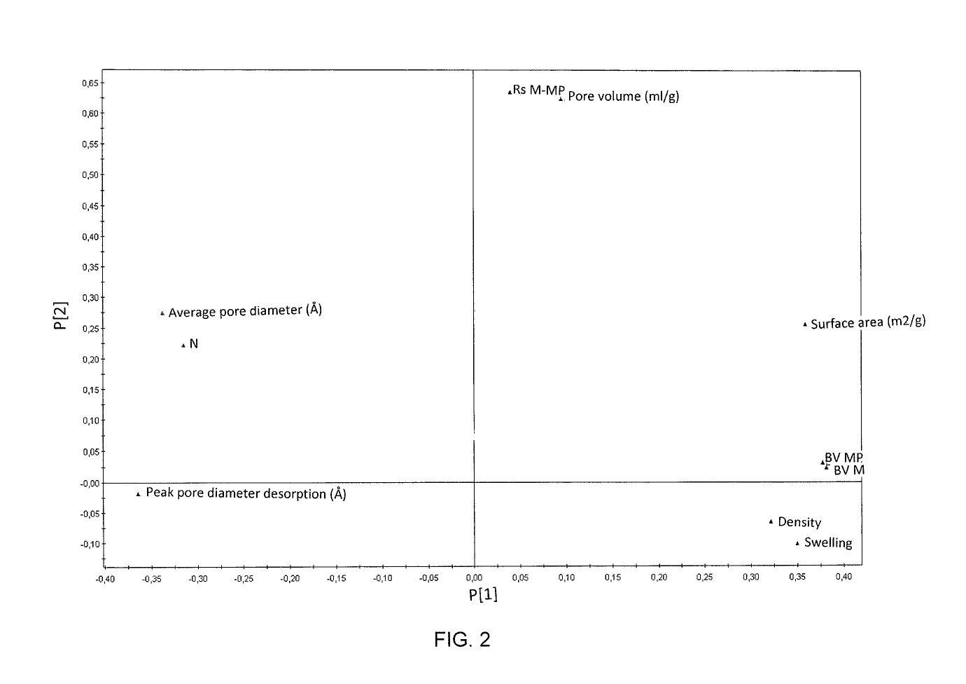

The relationship between certain physical parameters and separation performance of various resins or polymers was further investigated using principal component analysis (PCA). PCA is a statistical method that provides an overview of multidimensional data sets. Correlations between physical parameters such as specific surface area, specific pore volume, average pore diameter, peak pore diameter in BET desorption, swelling index and density and performance indicators such as resolution between monatin and monatin precursor (MP), plate number, number of bed volumes required for elution of monatin and MP are presented in FIG. 2. In this instance, the polymers had a 70-90 μm particle size although similar correlations may be observed with polymers having alternative particle sizes.

FIG. 2 is a loading plot that shows the relationship between the original variables and the principal components. The loading plot also shows correlations between the physical parameters of the resins (specific surface area, specific pore volume, average pore diameter, peak pore diameter in BET desorption, swelling index and density) and their separation performance (resolution between monatin and MP, plate number, number of bed volumes required for elution of monatin and MP). For instance, two variables that are close to each other in the loading plot have a positive correlation and two variables that are on opposite sides of the origin have a negative correlation. FIG. 2 shows a positive correlation between resolution and specific pore volume. Thus, in one aspect polymers with high specific pore volumes generally have high resolution. FIG. 2 also shows that there is a negative correlation between the number of bed volumes required for elution (or recovery) and pore size. Thus in another aspect, polymers with a large pore size generally require only a low number of bed volumes for elution. Based on this data, it has been identified that for a combination of high resolution with fast elution, the resin should have high specific pore volume and a large pore size.

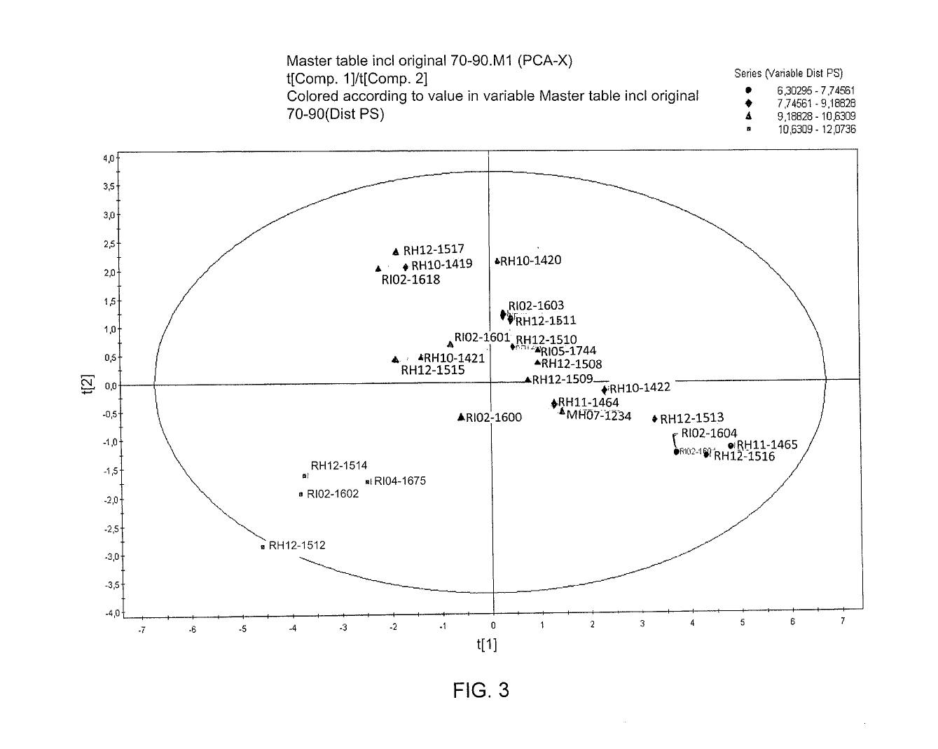

Additional data is available in FIG. 3, which is a score plot showing the distribution of a variety of different polymers. In FIG. 3, objects that are close to each other have similar properties. For instance, the polymers that combine high resolution with low number of bed volumes are located in the upper left of the plot. There are resins in the lower left corner of the plot which require an even lower number of bed volumes for elution, but these give lower resolutions. However, such resins may still be advantageous under circumstances where low elution volumes has a higher priority than high resolution.

Furthermore, it was found that the location of a resin in the score plot is related to the Skaarup distance between the resin and the solvent system that was used to produce the resin. The Skaarup distance was calculated using representative values for polystyrene (dispersion solubility parameter 19.2 MPa^{1/2}, polar solubility parameter 0.9 MPa^{1/2}, hydrogen bonding solubility parameter 2.1 MPa^{1/2}, source: CRC Handbook of polymer-liquid interaction parameters and solubility parameters p. 299) for the polystyrene/divinylbenzene copolymer. The resins that combine high resolution with a low number of bed volumes have Skaarup distances between 7.7 and 10.6 MPa^{1/2} and the resins that require only a very low number of bed volumes for elution but give lower resolution have Skaarup distances above MPa^{1/2}. Resins with Skaarup distances lower than 7.7 MPa^{1/2} generally have poor resolution and require a high number of bed volumes for elution.

The solubility behavior of an unknown substance may provide a clue as to its identity. The selection of solvents or solvent blends to satisfy such criterion is a fine art, based on experience, trial and error, and intuition guided by such rules of thumb as “like dissolves like” and various definitions of solvent “strength”. While such methods are suitable in many situations, an organized system is often needed that can facilitate the accurate prediction of complex solubility behavior. One such system is that provided by the Hansen Parameter. In 1936 Joel H. Hildebrand (who laid the foundation for solubility theory in his classic work on the solubility of nonelectrolytes in 1916) proposed the square root of the cohesive energy density as a numerical value indicating the solvency behavior of a specific solvent.

$$\partial = \sqrt{C} = {[ \frac{Δ H - RT}{{V}_{m}} ]}^{1 / 2} \qquad ( 2 )$$

Charles Hansen later developed a three parameter system which divided the total Hildebrand value into three parts: a dispersion force component, a hydrogen bonding component, and a polar component. These values are additive and can be represented by the following equation:

∂ₜ²=∂_{d}²+∂ₚ²+∂ₕ²

where
∂ₜ²=Total Hildebrand parameter
∂_{d}²=dispersion component
∂ₚ²=polar component
∂ₕ²=hydrogen bonding component

Charles Hansen also used a three-dimensional model (similar to that used by Crowley et al.) to plot polymer solubilities. He found that, by doubling the dispersion parameter axis, an approximately spherical volume of solubility would be formed for each polymer. This volume, being spherical, can be described in a simple way: the coordinates at the center of the solubility sphere are located by means of three component parameters (∂_{d}, ∂ₚ, ∂ₕ), and the radius of the sphere is indicated, called the interaction radius (R) (also referred to herein as the Skaarup distance (Ra)). The Hansen volume of solubility for a polymer is located within a 3-D model by giving the coordinates of the center of a solubility sphere (∂_{d}, ∂ₚ, ∂ₕ) and its radius of interaction (R). Liquids whose parameters lie within the volume are active solvents for that polymer. Stated another way, a polymer is probably soluble in a solvent (or solvent blend) if the Hansen parameters for the solvent lie within the solubility sphere for the polymer.

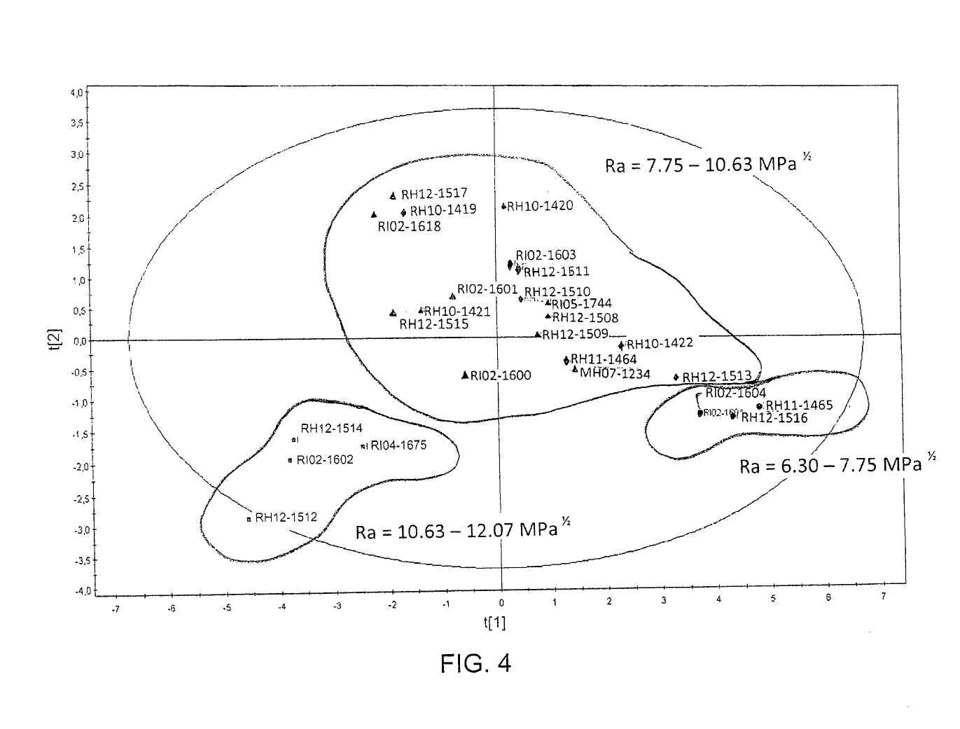

An additional benefit of using a solvent system within desired Hansen parameter space is that in addition to good porosity inside the particles, the particles themselves have an “open” structure, i.e. there is no skin on the surface that blocks access to the interior. This “open” structure is depicted by FIG. 4.

The Batches depicted in FIG. 3 are all styrene/divinylbenzene copolymers. Each of the polymers had a sieved particle size of 70-90 μm. Each of the polymers were also formed in the presence of a solvent system, the details of each solvent system being presented in Table 3a. In one aspect the porogen to monomer ratio was 1.85 meaning that for every 1 kg of monomer used, 1.85 kg of porogen was used. Batch #1 was formed in a 30 L reactor at a stirring rate of 300 rpm at 50° C. Batches 3-8 were formed in a 400 ml reactor at a stirring rate of 400 rpm at 50° C. and Batches 9-24 were formed in a 2000 ml reactor at a stirring rate of 200 rpm at 50° and Batches 25 was formed in a 2000 ml reactor at a stirring rate of 150 rpm at 50° C. with the exception that the stirring rate for Batch #25 was 150 rpm. Each of the Batches was also formed with the use of a polyvinylalcohol stabilizer. With the exception of Batch #1 (MH07-1234) where the percent concentration of the stabilizer was 0.67%, the percent concentration of the stabilizer was 0.5%. In one aspect, the copolymers were produced by suspension polymerization.

TABLE 3a
Batch Formulations

| | | | | | | | |
|---|---|---|---|---|---|---|---|
| 1 | MH07-1234 | Benzyl alcohol | 48.3 | Chloroform | 51.7 | — | 0 |
| 2 | RH10-1419 | Benzyl alcohol | 66 | Toluene | 22 | Methyl isobutyl ketone | 12 |
| 3 | RH10-1420 | Benzyl alcohol | 75 | Toluene | 25 | — | 0 |
| 4 | RH10-1421 | Benzyl alcohol | 81 | Toluene | 19 | — | 0 |
| 5 | RH10-1422 | Benzyl alcohol | 67 | Toluene | 33 | — | 0 |
| 6 | RH11-1464 | Benzyl alcohol | 37 | Toluene | 41 | 1-Pentanol | 22 |
| 7 | RH11-1465 | Benzyl alcohol | 44 | Toluene | 25 | Methyl isobutyl ketone | 31 |
| 8 | RH12-1508 | Benzyl alcohol | 43 | Chloroform | 45 | 1-Pentanol | 12 |
| 9 | RH12-1510 | Benzyl alcohol | 66 | Toluene | 22 | Methyl isobutyl ketone | 12 |
| 10 | RH12-1509 | Benzyl alcohol | 43 | Chloroform | 45 | 1-Pentanol | 12 |
| 11 | RH12-1511 | Benzyl alcohol | 66 | Toluene | 22 | Methyl isobutyl ketone | 12 |
| 12 | RH12-1512 | Benzyl alcohol | 55 | Chloroform | 20 | 1-Pentanol | 25 |
| 13 | RH12-1513 | Benzyl alcohol | 32 | Chloroform | 60 | 1-Pentanol | 8 |
| 14 | RH12-1514 | Benzyl alcohol | 25 | Chloroform | 40 | 1-Pentanol | 35 |
| 15 | RH12-1515 | Benzyl alcohol | 80 | Toluene | 15 | Methyl isobutyl ketone | 5 |
| 16 | RH12-1516 | Benzyl alcohol | 45 | Toluene | 40 | Methyl isobutyl ketone | 15 |
| 17 | RH12-1517 | Benzyl alcohol | 65 | Toluene | 10 | Methyl isobutyl ketone | 25 |
| 18 | RI02-1600 | Benzyl alcohol | 50 | Toluene | 0 | Methyl isobutyl ketone | 50 |
| 19 | RI02-1601 | Benzyl alcohol | 65 | Toluene | 10 | Methyl isobutyl ketone | 25 |
| 20 | RI02-1602 | Benzyl alcohol | 80 | Toluene | 5 | Methyl isobutyl ketone | 15 |
| 21 | RI02-1603 | Benzyl alcohol | 20 | Toluene | 5 | Methyl isobutyl ketone | 75 |
| 22 | RI02-1604 | Benzyl alcohol | 30 | Toluene | 40 | Methyl isobutyl ketone | 30 |
| 23 | RI02-1618* | Benzyl alcohol | 65 | Toluene | 10 | Methyl isobutyl ketone | 25 |
| 24 | RI04-1675 | Benzyl alcohol | 80 | Toluene | 5 | Methyl isobutyl ketone | 15 |
| 25 | RI05-1744 | Benzyl alcohol | 65 | Toluene | 10 | Methyl isobutyl ketone | 25 |

Additional data regarding the batches is also presented in Tables 3b-3d.

TABLE 3b
Physical and Surface Properties of Polymer Batches

| Batch | Descriptor | Rs M-MP | N | Rt M | BV M | Rt MP | BV MP | Rs M-I3P | Rt I3P | BV I3P |
|---|---|---|---|---|---|---|---|---|---|---|
| 1 | MH07-1234 | 1.22 | 249 | 30 | 3.1 | 17 | 1.7 | — | — | — |
| 2 | RH10-1419 | 1.51 | 357 | 27 | 2.7 | 16 | 1.6 | — | — | — |
| 3 | RH10-1420 | 1.48 | 381 | 31 | 3.1 | 18 | 1.8 | — | — | — |
| 4 | RH10-1421 | 1.25 | 303 | 24 | 2.4 | 15 | 1.5 | — | — | — |
| 5 | RH10-1422 | 1.37 | 221 | 36 | 3.6 | 19 | 1.9 | — | — | — |
| 6 | RH11-1464 | 1.08 | 109 | 28 | 2.8 | 17 | 1.7 | — | — | — |
| 7 | RH11-1465 | 1.15 | 79 | 44 | 4.4 | 23 | 2.3 | — | — | — |
| 8 | RH12-1508 | 1.27 | 168 | 31 | 3.1 | 18 | 1.8 | — | — | — |
| 9 | RH12-1510 | 1.32 | 243 | 31 | 3.1 | 18 | 1.8 | — | — | — |
| 10 | RH12-1509 | 1.25 | 227 | 30 | 3.0 | 18 | 1.8 | — | — | — |
| 11 | RH12-1511 | 1.38 | 348 | 29 | 2.9 | 17 | 1.7 | — | — | — |
| 12 | RH12-1512 | 0.92 | 391 | 18 | 1.8 | 13 | 1.3 | — | — | — |
| 13 | RH12-1513 | 1.30 | 239 | 38 | 3.8 | 20 | 2.0 | — | — | — |
| 14 | RH12-1514 | 1.14 | 312 | 21 | 2.1 | 14 | 1.4 | — | — | — |
| 15 | RH12-1515 | 1.17 | 302 | 24 | 2.4 | 15 | 1.5 | — | — | — |
| 16 | RH12-1516 | 1.12 | 87 | 44 | 4.4 | 23 | 2.3 | — | — | — |
| 17 | RH12-1517 | 1.45 | 419 | 26 | 2.6 | 16 | 1.6 | 1.69 | 52.0 | 5.2 |
| 18 | RI02-1600 | 1.20 | 210 | 26 | 2.6 | 16 | 1.6 | 1.53 | 57.0 | 5.7 |
| 19 | RI02-1601 | 1.33 | 328 | 27 | 2.7 | 16 | 1.6 | 1.24 | 57.0 | 5.7 |
| 20 | RI02-1602 | 1.09 | 389 | 19 | 1.9 | 13 | 1.3 | 1.54 | 35.0 | 3.5 |
| 21 | RI02-1603 | 1.29 | 201 | 30 | 3.0 | 18 | 1.8 | 1.58 | 63.0 | 6.3 |
| 22 | RI02-1604 | 1.13 | 152 | 36 | 3.6 | 19 | 1.9 | 1.85 | 79.0 | 7.9 |
| 23 | RI02-1618* | 1.37 | 389 | 25 | 2.5 | 15 | 1.5 | 1.74 | 51.0 | 5.1 |
| 24 | RI04-1675 | 1.15 | 340 | 22 | 2.2 | 14 | 1.4 | 1.56 | 43 | 4.3 |
| 25 | RI05-1744 | 1.42 | 331 | 29 | 2.9 | 17 | 1.7 | 1.71 | 60 | 6.0 |

TABLE 3c
Physical and Surface Properties of Polymer Batches

| | | | | BET data | | | | |
|---|---|---|---|---|---|---|---|---|
| | | | | | | Average | Peak pore | |
| | | | | Surface | Pore | pore | diameter | |
| | | Particle | | area | volume | diameter | desorption | |
| Batch | Descriptor | size | Swelling | (m²/g) | (ml/g) | (Å) | (Å) | Density |
| 1 | MH07-1234 | 80 | 1.4 | 369 | 1.2 | 131 | 400.00 | 0.3 |
| 2 | RH10-1419 | 78 | — | 306 | 1.4 | 183 | 570.00 | 0.22 |
| 3 | RH10-1420 | 83 | — | 422 | 1.5 | 142 | 490.00 | 0.22 |
| 4 | RH10-1421 | 81 | — | 351 | 1.4 | 155 | 650.00 | 0.23 |
| 5 | RH10-1422 | 84 | — | 404 | 1.1 | 108 | 260.00 | 0.26 |
| 6 | RH11-1464 | 84 | — | 413 | 1.5 | 145 | 400.00 | 0.27 |
| 7 | RH11-1465 | 85 | — | 450 | 1.2 | 105 | 190.00 | 0.30 |
| 8 | RH12-1508 | 81.5 | 1.3 | 397 | 1.4 | 143 | 510 | 0.26 |
| 9 | RH12-1510 | 80.8 | 1.3 | 378 | 1.4 | 146 | 630 | 0.26 |
| 10 | RH12-1509 | 82.9 | 1.3 | 368 | 1.3 | 142 | 470 | 0.27 |
| 11 | RH12-1511 | 81.1 | 1.3 | 373 | 1.4 | 151 | 460 | 0.28 |
| 12 | RH12-1512 | 82.7 | 1.1 | 169 | 0.7 | 165 | 950 | 0.20 |
| 13 | RH12-1513 | 84.2 | 1.4 | 410 | 1.1 | 102 | 260 | 0.33 |
| 14 | RH12-1514 | 82.4 | 1.2 | 193 | 0.8 | 172 | 1000 | 0.21 |
| 15 | RH12-1515 | 80.7 | 1.2 | 298 | 1.4 | 194 | 680 | 0.25 |
| 16 | RH12-1516 | 83.9 | 1.4 | 434 | 1.2 | 107 | 260 | 0.30 |
| 17 | RH12-1517 | 81.5 | 1.2 | 312 | 1.6 | 198 | 650 | 0.25 |
| 18 | RI02-1600 | 83.0 | 1.2 | 337 | 1.2 | 141 | 650 | — |
| 19 | RI02-1601 | 82.6 | 1.2 | 335 | 1.3 | 156 | 500 | — |
| 20 | RI02-1602 | 79.4 | 1.1 | 176 | 0.7 | 168 | 650 | — |
| 21 | RI02-1603 | 81.5 | 1.2 | 409 | 1.6 | 157 | 680 | — |
| 22 | RI02-1604 | 78.2 | 1.6 | 455 | 1.2 | 103 | 220 | — |
| 23 | RI02-1618* | 81.2 | 1.1 | 337 | 1.6 | 187 | 680 | — |
| 24 | RI04-1675 | 81 | 1.2 | 218 | 0.8 | 152 | 630 | — |
| 25 | RI05-1744 | 82.6 | 1.4 | 398 | 1.2 | 124 | 460 | — |

TABLE 3d
Hansen Solubility Characteristics of Polymer Batches

| | | Hansen parameters | | | Skaarup distance |
|---|---|---|---|---|---|
| Batch | Descriptor | ∂d | ∂p | ∂h | Dist PS |
| 1 | MH07-1234 | 18.1 | 4.9 | 10.3 | 9.34 |
| 2 | RH10-1419 | 17.8 | 5.1 | 9.4 | 8.84 |
| 3 | RH10-1420 | 18.3 | 4.9 | 10.3 | 9.28 |
| 4 | RH10-1421 | 18.3 | 5.2 | 11.2 | 10.20 |
| 5 | RH10-1422 | 18.3 | 4.5 | 9.4 | 8.33 |
| 6 | RH11-1464 | 17.6 | 4.1 | 8.7 | 7.93 |
| 7 | RH11-1465 | 17.2 | 4.9 | 7.2 | 7.63 |
| 8 | RH12-1508 | 17.8 | 5.1 | 10.9 | 10.16 |
| 9 | RH12-1510 | 17.8 | 5.1 | 9.4 | 8.84 |
| 10 | RH12-1509 | 17.8 | 5.1 | 10.9 | 10.16 |
| 11 | RH12-1511 | 17.8 | 5.1 | 9.4 | 8.84 |
| 12 | RH12-1512 | 17.5 | 5.7 | 12.6 | 12.07 |
| 13 | RH12-1513 | 17.8 | 4.7 | 9.7 | 8.95 |
| 14 | RH12-1514 | 17.1 | 5.2 | 11.5 | 11.16 |
| 15 | RH12-1515 | 18.1 | 5.4 | 11.1 | 10.27 |
| 16 | RH12-1516 | 17.7 | 4.2 | 7.0 | 6.63 |
| 17 | RH12-1517 | 17.4 | 5.7 | 9.6 | 9.54 |
| 18 | RI02-1600 | 16.6 | 6.2 | 8.3 | 9.60 |
| 19 | RI02-1601 | 17.4 | 5.7 | 9.6 | 9.54 |
| 20 | RI02-1602 | 17.8 | 6.0 | 11.2 | 10.83 |
| 21 | RI02-1603 | 15.9 | 5.9 | 5.5 | 8.92 |
| 22 | RI02-1604 | 17.2 | 4.2 | 5.7 | 6.30 |
| 23 | RI02-1618* | 17.4 | 5.7 | 9.6 | 9.54 |
| 24 | RI04-1675 | 17.8 | 6.0 | 11.2 | 10.83 |
| 25 | RI05-1744 | 17.4 | 5.7 | 9.6 | 9.54 |

In one embodiment, the polymer described herein is an adsorbent resin with a polystyrene-divinylbenzene matrix and without functionalized groups having the properties listed in Table 4. As used herein, particle size values are diameters.

TABLE 4
Polymer Properties

| | Limit | |
|---|---|---|
| Particle Size | | |
| Particle size average | 85-100 | μm |
| Particle size distribution | 50-150 μm > 96% | |
| Porosity | | |
| Specific surface area | 300-475 | m²/g |
| Specific pore volume | 0.9-1.3 | g/ml |
| Average pore diameter | 85-150 | Angstrom |

In an alternative embodiment, the polymer described herein is an adsorbent resin with a polystyrene-divinylbenzene matrix and without functionalized groups having the properties listed in Table 5.

TABLE 5
Polymer Properties

| | Limit | |
|---|---|---|
| Particle Size | | |
| Particle size average | 85-100 | μm |
| Particle size distribution | 50-150 μm > 96% | |
| Porosity | | |
| Specific surface area | 100-500 | m²/g |
| Specific pore volume | 0.5-1.8 | g/ml |
| Average pore diameter | 50-250 | Angstrom |

It is recognized that reverse phase resins having properties outside the parameters shown in Tables 4 and 5 may be used in the method and system described herein for separation and purification of monatin. For example, in one aspect described herein is an adsorbent resin with a polystyrene-divinylbenzene matrix and without functionalized groups having properties listed in Table 6.

TABLE 6
Polymer Properties

| | Limit | |
|---|---|---|
| Particle Size | | |
| Particle size average | 35-45 | μm |
| Particle size distribution | 30-50 μm > 95% | |
| Porosity | | |
| Specific surface area | 250-700 | m²/g |
| Specific pore volume | 0.5-1.8 | g/ml |
| Average pore diameter | 50-250 | Angstrom |

It would be within the knowledge of one skilled in the art to identify appropriate monomers to use as well as usage amounts to obtain the necessary properties such as those identified in Table 6. For instance, where a smaller particle size average and particle size distribution is desired to create greater surface area, the monomers styrene and divinylbenzene may be used in various ratios; where the amount of styrene may range from about 0.01 to 80% and the amount of divinylbenzene may range from about 20-99.99%. One of skill in the art would also recognize that commercial sources of divinylbenzene may not be 100% pure and may contain other monomers. For example, in one aspect the commercially available divinylbenzene contains about 80% divinylbenzene and about 19% other monomers where the other monomers include diethylenebenzene and styrene.

It has been further discovered based on the data in FIGS. 2 and 3 that the location of a polymer on the score plot is related to the Skaarup distance (Ra) between the polymer and the solvent system that was used to produce the resin, where the Skaarup distance (Ra) is calculated herein as

Ra=(4(∂d2−δd1)²+(δ2−δp1)²+(δH2−δH1)²)^{1/2}; where

δd1 is the dispersion solubility parameter, δp1 is the polar solubility parameter and δh1 is the hydrogen bonding solubility parameter for the solvent system and δd2 is the dispersion solubility parameter, δp2 is the polar solubility parameter and δh2 is the hydrogen solubility parameter for the polymer. The identification of this relationship makes it possible to better predict and generate polymers with desired properties and functionality as well as identify solvents for production of such polymers based upon Hansen Solubility Parameters such as the Skaarup number. For instance, the polymer identified as Batch #1 or MH07-1234 in FIG. 2-3 and Tables 3a-3d was produced by suspension polymerization using the solvents chloroform and benzyl alcohol. In addition to the properties listed in Tables 3b-3c, this particular polymer has proven to be effective at recovering monatin at levels of greater than 90; and at levels of greater than 95%. It may be desirable, however, to identify alternative solvents to use in the production of a polystyrene/divinylbenzene copolymer, though. By using Hansen Parameters such as the solubility parameters and Ra, an alternative solvent system may be identified to generate a polymer having similar performance characteristics.

For example, the Skaarup distance may be calculated using representative values for polystyrene (dispersion solubility parameter 19.2 MPa^{1/2}, polar solubility parameter 0.9 MPa^{1/2}, hydrogen bonding solubility parameter 2.1 MPa^{1/2}, source: CRC Handbook of polymer-liquid interaction parameters and solubility parameters p. 299) for a polystyrene/divinylbenzene copolymer. Referring to FIG. 3, the polymers that combine high resolution with a low number of bed volumes have Skaarup distances between 7.7 and 10.9 MPa^{1/2} and the polymers that require only a very low number of bed volumes for elution but give lower resolution have Skaarup distances above 10.6 MPa^{1/2}. Polymers with Skaarup distances lower than 7.7 MPa^{1/2} generally have poor resolution and require a high number of bed volumes for elution. FIG. 4 has been provided to highlight these general relationships. In this example, the solvent system may also be selected such that it has a dispersion solubility parameter between 15.9 and 18.3 MPa^{1/2}, a polar solubility parameter between 4.0 and 6.2 MPa^{1/2} and a hydrogen bonding solubility parameter between 5.5 and 12.7 MPa^{1/2}. For modeling of multi-component solvent systems, it may be assumed that there is a linear relationship between the individual solubility parameters and the mixture. For example, if you have a two component system of solvent A and B:

D(blend)=[volume fraction (A)*D(A)]+[volume fraction (B)*D(B)]→Dispersion parameter for blend

P(blend)=[volume fraction (A)*P(A)]+[volume fraction (B)*P(B)]→Polar parameter for blend

H(blend)=[volume fraction (A)*H(A)]+[volume fraction (B)*H(B)]→Hydrogen parameter for blend.

Thus, in one embodiment is a method of preparing a polymer in the presence of a solvent system, where the solvent system is selected such that it has a dispersion solubility parameter between about 15.9 and 18.3 MPa^{1/2}, a polar solubility parameter between about 4.0 and 6.2 MPa^{1/2} and a hydrogen bonding solubility parameter between about 5.5 and 12.7 MPa^{1/2}. The method may further include selecting a solvent system such that the polymer and the solvent system have a Skaarup distance (Ra) between the polymer and the solvent system of from about 7.7 MPa^{1/2} to 10.9 MPa^{1/2}. Alternatively, the Skaarup distance (Ra) between the polymer and the solvent system is from about 9 MPa^{1/2} to 10.6 MPa^{1/2}. In an additional aspect, the polymer may have a specific pore volume between about 0.5 mL/g and 1.8 mL/g. Alternatively, the polymer may have a specific pore volume greater than about 1 mL/g. In yet another aspect, the polymer may have a specific surface area between about 100 m²/g and 500 m²/g. In still another aspect, the polymer may have a specific surface area between about 100 m²/g and 700 m²/g. Alternatively, the polymer may have a specific pore volume between about 0.5 mL/g and 1.8 mL/g and a specific surface area of between about 100 m²/g and 500 m²/g. In yet another aspect, the polymer may have a specific pore volume between about 0.5 mL/g and 1.8 mL/g and a specific surface area of between about 100 m²/g and 700 m²/g. The polymer may also be characterized by its average pore diameter being calculated herein as

average pore diameter=40,000*(specific pore volume)/(specific surface area),

where the average pore diameter is in Angstroms, the specific pore volume is in mL/g and the specific surface area is in m²/g. In one aspect, the polymer has an average pore diameter between about 50 Angstroms to 450 Angstroms. In another aspect, the polymer has an average pore diameter between about 50 Angstroms to 250 Angstroms. And in yet another aspect, polymer has an average pore diameter between about 100 Angstroms to 250 Angstroms. In a particular aspect, the polymer is a polystyrene/divinylbenzene copolymer.

There are many solvent combinations that will generate parameters within the desired Hansen space and any solvent system that provides Hansen parameters within the desired Hansen space is contemplated. For example, in one embodiment the solvent system is selected from chloroform, benzyl alcohol, 1-pentanol, ethyl acetate, toluene, 1-decanol, methyl isobutyl ketone and combinations thereof. Other similar solvent types could be used, however, such as octanol, dodecanol, decanol are aliphatic alcohols as a substitute for benzyl alcohol. Likewise, toluene may be substituted with aliphatic hydrocarbons, aromatic solvents such as xylene and methyl isobutyl ketone could be substituted with another ketone having similar hansen parameters. Other acceptable solvent classes include ethers, esters and solvents of combined functionality (both ether and alcohol, phenols, and difunctional alcohols).

In one aspect is a two component system with 1-octanol and chloroform. The volume fraction of 1-octanol to chloroform may be about 0.48-1.0, alternatively about 0.75-1.0. In an alternative aspect is a two component system with benzyl alcohol and chloroform. In this aspect, benzyl alcohol may be present in the range of about 25 to 82% (by volume). In another aspect is a three component system with benzyl alcohol, toluene and methyl isobutyl ketone. In one aspect enzyl alcohol is present at about 15-92% (by volume), toluene is present at about 0-45% (by volume) and methyl isobutyl ketone is present at about 0-80% (by volume). In a further aspect, the solvent system includes 80% (by weight) benzyl alcohol, 5% (by weight) toluene and 15% (by weight) methyl isobutyl ketone.

It would be within the knowledge of one skilled in the art to identify appropriate solvents to use as well as usage amounts based upon factors such as the process for preparing the polymer. For instance, where suspension polymerization is used, water miscible solvents would be excluded because of the nature of the process. Furthermore, certain solvent combinations could give rise to different types of considerations such as negative physical properties of the formed polymer or process related problems such as poor stabilization of the suspended droplets/particles during polymerization.

The polymer may be adapted to recover monatin from a monatin-containing mixture where the monatin-containing mixture may include monatin, monatin precursor (MP) and I3P. The polymer may further have a resolution of greater than about 0.7 between monatin and monatin precursor. Additionally, the polymer may have an elution volume for monatin of less than 5 bed volumes with recovery greater than 95%.

In a further aspect, the polymer has a swelling index of less than 1.3 of polymer wetted by ethanol to dry polymer. It is generally preferred to have a low swelling index, such as below about 1.1 to 1.3 to provide certain benefits such making it easier to clean the containment vessel of the polymer (i.e. allows for clean in place), switching between different solvents and maintaining more consistent packing of the resin in the containment vessel.

In another embodiment is a method of preparing a polymer in the presence of a solvent system, where the solvent system is selected such that the polymer has an average pore diameter between about 50 Angstroms to 450 Angstroms. In one aspect, the polymer has an average pore diameter between about 50 Angstroms to 250 Angstroms. And in yet another aspect, polymer has an average pore diameter between about 100 Angstroms to 250 Angstroms. In a particular aspect, the polymer is a polystyrene/divinylbenzene copolymer.

In an additional aspect, the polymer may have a specific pore volume between about 0.5 mL/g and 1.8 mL/g. Alternatively, the polymer may have a specific pore volume greater than about 1 mL/g. In yet another aspect, the polymer may have a specific surface area between about 100 m²/g and 500 m²/g. In still another aspect, the polymer may have a specific surface area between about 100 m²/g and 700 m²/g. Alternatively, the polymer may have a specific pore volume between about 0.5 mL/g and 1.8 mL/g and a specific surface area of between about 100 m²/g and 500 m²/g. In yet another aspect, the polymer may have a specific pore volume between about 0.5 mL/g and 1.8 mL/g and a specific surface area of between about 100 m²/g and 700 m²/g.

The method may further include selecting a solvent system such that the polymer and the solvent system have a Skaarup distance (Ra) between the polymer and the solvent system of from about 7.7 MPa^{1/2} to 10.9 MPa^{1/2}. Alternatively, the Skaarup distance (Ra) between the polymer and the solvent system is from about 9 MPa^{1/2} to 10.6 MPa^{1/2}.

As previously discussed, there are many solvent combinations that will generate parameters within the desired Hansen space and any solvent system that provides Hansen parameters within the desired Hansen space is contemplated. For example, in one embodiment the solvent system is selected from chloroform, benzyl alcohol, 1-pentanol, ethyl acetate, toluene, 1-decanol, methyl isobutyl ketone and combinations thereof. In one aspect is a two component system with 1-octanol and chloroform. The volume fraction 1-octanol to chloroform may be about 0.48-1.0, alternatively about 0.75-1.0. In an alternative aspect is a two component system with benzyl alcohol and chloroform. In this aspect, benzyl alcohol may be present in the range of about 25 to 82% (by volume). In another aspect is a three component system with benzyl alcohol, toluene and methyl isobutyl ketone. In one aspect benzyl alcohol is present at about 15-92% (by volume), toluene is present at about 0-45% (by volume) and methyl isobutyl ketone is present at about 0-80% (by volume). In a further aspect, the solvent system includes 80% (by weight) benzyl alcohol, 5% (by weight) toluene and 15% (by weight) methyl isobutyl ketone.

Similar to the previous embodiment, the polymer may also be adapted to recover monatin from a monatin-containing mixture where the monatin-containing mixture may include monatin, monatin precursor (MP) and I3P. The polymer may further have a resolution of greater than about 0.7 between monatin and monatin precursor. Additionally, the polymer may have an elution volume for monatin of less than about 5 bed volumes with recovery greater than about 95%.

In a further aspect, the polymer has a swelling index of less than about 1.3 of polymer wetted by ethanol to dry polymer. It is generally preferred to have a low swelling index, such as below about 1.1 to 1.3 to provide certain benefits such making it easier to clean the containment vessel of the polymer (i.e. allows for clean in place), switching between different solvents and maintaining more consistent packing of the resin in the containment vessel.

In a further embodiment is a polymer adapted to recover monatin from a mixture, where the polymer has an average pore diameter between about 50 Angstroms to 450 Angstroms. In one aspect, the polymer has an average pore diameter between about 50 Angstroms to 250 Angstroms. And in yet another aspect, polymer has an average pore diameter between about 100 Angstroms to 250 Angstroms. In a particular aspect, the polymer is a polystyrene/divinylbenzene copolymer.

In an additional aspect, the polymer may have a specific pore volume between about 0.5 mL/g and 1.8 mL/g. Alternatively, the polymer may have a specific pore volume greater than about 1 mL/g. In yet another aspect, the polymer may have a specific surface area between about 100 m²/g and 500 m²/g. Alternatively, the polymer may have a specific pore volume between about 0.5 mL/g and 1.8 mL/g and a specific surface area of between about 100 m²/g and 500 m²/g.

In yet another aspect, the polymer may have a specific pore volume between about 0.5 mL/g and 1.8 mL/g. Alternatively, the polymer may have a specific pore volume greater than about 1 mL/g. In yet another aspect, the polymer may have a specific surface area between about 250 m²/g and 700 m²/g. Alternatively, the polymer may have a specific pore volume between about 0.5 mL/g and 1.8 mL/g and a specific surface area of between about 250 m²/g and 700 m²/g.

In some aspects, the polymer is adapted to recover monatin from a monatin-containing mixture such that the recovered monatin has a purity level greater than about 90%. In a particular aspect, the monatin-containing mixture includes monatin, monatin precursor and I3P. In other aspects, the polymer is adapted to recover monatin from the mixture such that the recovered monatin has a purity level greater than 95%.

The polymer may further have swelling index of less than about 1.3.

In yet another embodiment is a method of recovering monatin from a mixture. The method may include the steps of using a polymer made in the presence of a solvent system selected such that the solvent system has a dispersion solubility parameter between about 15.9 and 18.3 MPa^{1/2}, a polar solubility parameter between about 4.0 and 6.2 MPa^{1/2} and a hydrogen bonding solubility parameter between about 5.5 and 12.7 MPa^{1/2}. In a further aspect, the solvent system is selected such that the Skaarup distance (Ra) between the polymer and the solvent system of from about 7.7 MPa^{1/2} to 10.9 MPa^{1/2}. Alternatively, the solvent system is selected such that the Skaarup distance (Ra) between the polymer and the solvent system is from about 9 MPa^{1/2} to 10.6 MPa^{1/2}. Acceptable solvent systems are consistent with those previously described.

In an additional aspect, the polymer may have a specific pore volume between about 0.5 mL/g and 1.8 mL/g. Alternatively, the polymer may have a specific pore volume greater than about 1 mL/g. In yet another aspect, the polymer may have a specific surface area between about 100 m²/g and 500 m²/g. In still another aspect, the polymer may have a specific surface area between about 100 m²/g and 700 m²/g. Alternatively, the polymer may have a specific pore volume between about 0.5 mL/g and 1.8 mL/g and a specific surface area of between about 100 m²/g and 500 m²/g. In still another aspect, the polymer may have a specific pore volume between about 0.5 mL/g and 1.8 mL/g and a specific surface area of between about 100 m²/g and 700 m²/g.

In one aspect, the polymer has an average pore diameter between about 50 Angstroms to 450 Angstroms. In another aspect, the polymer has an average pore diameter between about 50 Angstroms to 250 Angstroms. And in yet another aspect, polymer has an average pore diameter between about 100 Angstroms to 250 Angstroms. In a particular aspect, the polymer is a polystyrene/divinylbenzene copolymer.

In some aspects, the polymer is adapted to recover monatin from a monatin-containing mixture such that the recovered monatin has a purity level greater than about 90%. In a particular aspect, the monatin-containing mixture includes monatin, monatin precursor and I3P. In other aspects, the polymer is adapted to recover monatin from the mixture such that the recovered monatin has a purity level greater than about 95%. Additionally, the polymer may have a resolution greater than about 0.7 between monatin and monatin precursor. The polymer may further have swelling index of less than about 1.3. In another aspect, the polymer has an elution volume for monatin of less than about 5 bed volumes with recovery greater than about 95%.

In some embodiments using a DAC column in combination with a reverse phase resin, the following operating conditions may be used: An oxygen free environment is maintained due to instability of one or more intermediates (for example, I3P) in the presence of oxygen. As such, before starting the DAC column, the feed and elution tanks may be sparged with nitrogen and then during operation, it may be kept under a nitrogen overlay. The temperature inside the DAC column may be maintained at an operating temperature between about 10 and about 30 degrees Celsius. In some embodiments, the temperature may be maintained at less than about 25 degrees Celsius. In other embodiments, the temperature is maintained between about 10 and about 18 degrees Celsius; in yet other embodiments, the temperature is maintained at about 15 degrees Celsius. The pH inside the DAC column prior to injection may be maintained between about 5.0 and about 9.0 depending, in part, on the pH and ionic strength of the eluent chosen.

Additional aspects of the invention are illustrated in the following non-limiting examples.

### EXAMPLES

### Example 1

### Evaluation of Polymers

The polymers reported in Tables 1 and 2 were evaluated by chromatography in a 150 mm length×4.6 mm inner diameter column with a mobile phase consisting of 5% ethanol in 10 mM phosphate buffer, pH 7.25 and a flow of 0.25 mL/min. The resolution (Rs) between MP and monatin was determined by injecting 20 pt of a 0.5 mg/mL sample of MP and monatin precursor in water. The resolution (Rs) between monatin and I3P was determined by injecting 20 μL of a 0.33 mg/mL sample of MP, monatin and I3P in water. The number of bed volumes (BV) required for elution was calculated from the retention time, flow and bed volume. The plate number (N) was determined by injecting 5 μL of a 1 mg/mL of NaNO₂ in water and measuring the peak width. The swelling index was measured by adding ethanol to a dry sample of resin and calculated as (swelling index)=(volume of wet resin)/(volume of dry resin).

### Example 2

### Preparation of Polymer

The following is a representative recipe and procedure for preparation of a reverse phase polymer.

### Batch Size=2846 g

### Reactor Volume=30 L

### RPM=300

| | | | | Active | | |
|---|---|---|---|---|---|---|
| | | | Mw | Content) | % | |
| Temperature = 50° C. | | M (g) | (g/mol) | %) | (on Monomer) | Supplier |
| Continuous Phase | | | | | | |
| Media | Water | 17692.31 | 18 | 0 | 621.62 | AB |
| Stabilizer | Polyvinyl | 120.00 | — | 100 | 4.22 | Celanese |
| | alcohol | | | | | |
| | Celvol 523 | | | | | |
| SUM Continuous Phase | | 17812.31 | | | | |
| Organic Phase | | | | | | |
| Solvent | Benzyl | 3215.38 | 108.14 | | 112.97 | SA |
| | Alcohol | | | | | |
| Solvent | Chloroform | 3436.92 | 119.38 | | 120.76 | SA |
| Monomer | DVB | 1406.15 | 130.19 | 80 | 49.41 | SA |
| Monomer | Styrene | 1393.85 | 104.15 | | 48.97 | SA |
| Initiator | ABDV | 46.15 | 248.37 | | 1.62 | WAKO |

| | |
|---|---|
| Sum Organic Phase | 9498.45 |

Materials: Polyvinyl alcohol, trade name Celvol 523 was purchased from celanese Benzylalcohol (cas 100-51-6,30-100%) was purchased from Swedhandling. Chloroform (cas 67-66-3,>99%), divinylbenzene (cas 1321-74-0, technical 80%), Styrene (cas 100-42-5,>99%) were obtained from Sigma-aldrich. ABDV (initiator) (2.2′-Azobis(2,4-dimethylvaleronitril) (cas 4419-11-8) was obtained from Wako chemicals. All chemicals were used as received without further purification.

Procedure: Celvol 523 was dissolved in distilled water by stirring % 80° C. for 2 h. Benzylalcohol, chloroform, styrene and divinylbenzene were mixed and then initiator ABDV was added and dissolved by stirring. The organic phase was then added to water phase and stirred @ ambient temperature for 1 h. reaction temperature=50° C. for 16 h (overnight) and then increased to 70° C. for 2 h. Stirring rate was 300 rpm with an anchor type stirrer. The final suspension was filtered, washed & dried in a lab-scale nutsch type filter. The polymer beads were classified by wet-sieving with a cut-off @ 50 μm.

### Exemplary Embodiments

A. A method of preparing a polymer, wherein the polymer is made in the presence of a solvent system, and wherein the solvent system is selected such that it has a dispersion solubility parameter between 15.9 and 18.3 MPa^{1/2}, a polar solubility parameter between 4.0 and 6.2 MPa^{1/2} and a hydrogen bonding solubility parameter between 5.5 and 12.7 MPa^{1/2}.
B. The method of embodiment A, wherein the solvent system is selected such that the polymer and the solvent system have a Skaarup distance (Ra) between the polymer and the solvent system of from 7.7 MPa^{1/2} to 10.9 MPa^{1/2}.
C. The method of embodiment B, wherein the Skaarup distance (Ra) between the polymer and the solvent system is from 9 MPa^{1/2} to 10.6 MPa^{1/2}
D. The method of embodiments A-C, wherein the polymer has a specific pore volume between 0.5 mL/g and 1.8 mL/g.
E. The method of embodiments A-D, wherein the polymer has a specific pore volume greater than 1 mL/g
F. The method of embodiments A-E, wherein the polymer has a specific surface area between 100 m²/g and 500 m²/g.
G. The method of embodiments A-C, wherein the polymer has a specific pore volume between 0.5 mL/g and 1.8 mL/g and a specific surface area of between 100 m²/g and 500 m²/g.
H. The method according to any of the preceding embodiments, wherein the polymer has an average pore diameter between 50 Angstroms to 450 Angstroms, where the average pore diameter is calculated as

Average Pore Diameter=40,000*(specific pore volume)/(specific surface area) and

where
the average pore diameter is in Angstroms, the specific pore volume is in mL/g and the specific surface area is in m²/g.
I. The method of embodiments H, wherein the polymer has an average pore diameter between 50 Angstroms to 250 Angstroms.
J. The method of embodiment I, wherein the polymer has an average pore diameter between 100 Angstroms to 250 Angstroms.
K. The method according to any of the preceding embodiments, wherein the polymer is a polystyrene/divinylbenzene copolymer.
L. The method according to any of the preceding embodiments, wherein the solvent system comprises a solvent selected from the group consisting of chloroform, benzyl alcohol, 1-pentanol, ethyl acetate, toluene, 1-decanol, methyl isobutyl ketone or combinations thereof.
M. The method according to any of the preceding embodiments, wherein the solvent system is a two component system.
N. The method of embodiment M, wherein the two components are chloroform and benzyl alcohol.
O. The method according to any of the preceding embodiments, wherein the solvent system is a three component system.
P. The method of embodiment O, wherein the three components are benzyl alcohol, toluene and methyl isobutyl ketone.
Q. The method according to any of the preceding embodiments, wherein the polymer is adapted to recover monatin from a mixture.
R. The method of embodiment Q, wherein the mixture comprises monatin, monatin precursor and I3P.
S. The method of embodiment R, wherein the polymer has a resolution of greater than 0.7 between monatin and monatin precursor.
T. The method of embodiments R or S, wherein the polymer has an elution volume for monatin of less than 5 bed volumes with recovery greater than 95%.
U. The method according to any of the preceding embodiments, wherein the polymer has a swelling index of less than 1.3 of polymer wetted by ethanol to dry polymer.
V. A polymer according to the method of embodiments A-U.
W. A method of preparing a polymer, wherein the polymer is made in the presence of a solvent system, and wherein the solvent system is selected such that the polymer has an average poor diameter between 50 Angstroms to 450 Angstroms, where the average pore diameter is calculated as

Average Pore Diameter=40,000*(specific pore volume)/(specific surface area) and

where
the average pore diameter is in Angstroms, the specific pore volume is in mL/g and the specific surface area is in m²/g.
X. The method of embodiment W, wherein the solvent system is selected such that the polymer and the solvent system have a Skaarup distance (Ra) between the polymer and the solvent system of from 7.7 MPa^{1/2} to 10.9 MPa^{1/2}.
Y. The method of embodiment X, wherein the Skaarup distance (Ra) between the polymer and the solvent system is from 9 MPa^{1/2} to 10.6 MPa^{1/}
Z. The method of embodiments W or X, wherein the polymer has a specific pore volume between 0.5 mL/g and 1.8 mL/g.
AA. The method of embodiments W or X, wherein the polymer has a specific pore volume greater than 1 mL/g
BB. The method of embodiments W or X, wherein the polymer has a specific surface area between 100 m²/g and 500 m²/g
CC. The method of embodiments W or X, wherein the polymer has a specific pore volume between 0.5 mL/g and 1.8 mL/g and a specific surface area of between 100 m²/g and 500 m²/g.
DD. The method of embodiments W-CC, wherein the polymer has an average pore diameter between 50 Angstroms to 250 Angstroms.
EE. The method of embodiments W-CC, wherein the polymer has an average pore diameter between 100 Angstroms to 200 Angstroms.
FF. The method according to embodiments W-EE, wherein the solvent system comprises a solvent selected from the group consisting of chloroform, benzyl alcohol, 1-pentanol, ethyl acetate, toluene, 1-decanol, methyl isobutyl ketone or combinations thereof.
GG. The method according to embodiments W-FF, wherein the solvent system is a two component system.
HH. The method of embodiment GG, wherein the two components are chloroform and benzyl alcohol.
II. The method according to embodiments W-FF, wherein the solvent system is a three component system.
JJ. The method of embodiment II, wherein the three components are benzyl alcohol, toluene and methyl isobutyl ketone.
KK. The method according to embodiments W-JJ, wherein the polymer is adapted to recover monatin from a mixture.
LL. The method of embodiment KK, wherein the mixture comprises monatin, monatin precursor and I3P.
MM. The method of embodiment LL, wherein the polymer has a resolution of greater than 0.7 between monatin and monatin precursor.
NN. The method of embodiments LL or MM, wherein the polymer has an elution volume for monatin of less than 5 bed volumes with recovery greater than 95%.
OO. The method of embodiments W-NN, wherein the polymer is a polystyrene/divinylbenzene copolymer.
PP. The method of embodiments W-OO, wherein the polymer has a swelling index of less than 1.3.
QQ. A polymer according to the method of embodiments W-PP.
RR. A polymer adapted to recover monatin from a mixture, wherein the polymer has an average pore diameter between 50 Angstroms to 450 Angstroms, where the average pore diameter is calculated as

Average Pore Diameter=40,000*(specific pore volume)/(specific surface area) and

where
the average pore diameter is in Angstroms, the specific pore volume is in mL/g and the specific surface area is in m²/g.
SS. The polymer of embodiment RR, wherein the specific pore volume is greater than 1 ml/g.
TT. The polymer of embodiment RR, wherein the polymer has a specific pore volume between 0.5 mL/g and 1.8 mL/g.
UU. The polymer of embodiment RR, wherein the polymer has a specific surface area between 100 m²/g and 500 m²/g.
VV. The polymer of embodiment RR, wherein the polymer has a specific pore volume between 0.5 mL/g and 1.8 mL/g and a specific surface area of between 100 m²/g and 500 m²/g.
WW. The polymer of embodiments RR-VV, wherein the polymer has an average poor diameter between 50 Angstroms to 250 Angstroms.
XX. The polymer of embodiments RR-VV, wherein the polymer has an average poor diameter between 100 Angstroms to 200 Angstroms.
YY. The polymer of embodiments RR-XX, wherein the polymer is a polystyrene/divinylbenzene copolymer.
ZZ. The polymer of embodiments RR-YY, wherein the polymer is adapted to recover monatin from the mixture such that the recovered monatin has a purity level greater than 90%.
AAA. The polymer of embodiments RR-ZZ, wherein the polymer is adapted to recover monatin from the mixture such that the recovered monatin has a purity level greater than 95%.
BBB. The polymer of embodiments RR-AAA, wherein the polymer has a swelling index of less than 1.3.
CCC. A method of recovering monatin from a mixture, comprising using a polymer made in the presence of a solvent system, and wherein the solvent system is selected such that it has a dispersion solubility parameter between 15.9 and 18.3 MPa^{1/2}, a polar solubility parameter between 4.0 and 6.2 MPa^{1/2} and a hydrogen bonding solubility parameter between 5.5 and 12.7 MPa^{1/2}.
DDD. The method of embodiment CCC, wherein the solvent system is selected such that the polymer and the solvent system have a Skaarup distance (Ra) between the polymer and the solvent system of from 7.7 MPa^{1/2} to 10.9 MPa^{1/2}.
EEE. The method of claim 56, wherein the Skaarup distance (Ra) between the polymer and the solvent system is from 9 MPa^{1/2} to 10.6 MPa^{1/2}.
FFF. The method of embodiments CCC-EEE, wherein the polymer has an average pore diameter between 50 Angstroms to 450 Angstroms, where the average pore diameter is calculated as

Average Pore Diameter=40,000*(specific pore volume)/(specific surface area) and

where
the average pore diameter is in Angstroms, the specific pore volume is in mL/g and the specific surface area is in m²/g.
GGG. The method of embodiment FFF, wherein the polymer has a specific pore volume between 0.5 mL/g and 1.8 mL/g.
HHH. The method of embodiment FFF, wherein the polymer has a specific pore volume greater than 1 mL/g
III. The method of embodiments CCC-HHH, wherein the polymer has a specific surface area between 100 m²/g and 500 m²/g.
JJJ. The method of embodiments CCC-III, wherein the polymer has a specific pore volume between 0.5 mL/g and 1.8 mL/g and a specific surface area of between 100 m²/g and 500 m²/g.
KKK. The method of embodiments CCC-JJJ, wherein the polymer has an average pore diameter between 50 Angstroms to 250 Angstroms.
LLL. The method of embodiments CCC-JJJ, wherein the polymer has an average pore diameter between 100 Angstroms to 200 Angstroms.
MMM. The method according to embodiments CCC-LLL, wherein the polymer is a polystyrene/divinylbenzene copolymer.
NNN. The method according to embodiments CCC-LLL, wherein the solvent system comprises a solvent selected from the group consisting of chloroform, benzyl alcohol, 1-pentanol, ethyl acetate, toluene, 1-decanol, methyl isobutyl ketone or combinations thereof.
OOO. The method of embodiment CCC-NNN, wherein the solvent system is a two component system.
PPP. The method of embodiment OOO, wherein the two components are chloroform and benzyl alcohol.
QQQ. The method of embodiments CCC-NNN, wherein the solvent system is a three component system.
RRR. The method of embodiment QQQ, wherein the three components are benzyl alcohol, toluene and methyl isobutyl ketone.
SSS. The method of embodiments CCC-RRR, wherein the mixture comprises monatin, monatin precursor and I3P.
TTT. The method of embodiments CCC-SSS, wherein the polymer has a resolution of greater than 0.7 between monatin and monatin precursor.
UUU. The method of embodiments CCC-TTT, wherein the polymer has an elution volume for monatin of less than 5 bed volumes with recovery greater than 95%.
VVV. The method of embodiments CCC-UUU, wherein the polymer is adapted to recover monatin from the mixture such that the recovered monatin has a purity level greater than 90%.
WWW. The method of embodiment VVV, wherein the polymer is adapted to recover monatin from the mixture such that the recovered monatin has a purity level greater than 95%.
XXX. The method of embodiments CCC-WWW, wherein the polymer is a polystyrene/divinylbenzene copolymer.
YYY. The method of embodiments CCC-XXX, wherein the polymer has a swelling index of less than 1.3.

What is claimed is:

1. A polymer adapted to recover monatin from a mixture, wherein the polymer has an average pore diameter between 50 Angstroms to 450 Angstroms, where the average pore diameter is calculated as
Average Pore Diameter=40,000*(specific pore volume)/(specific surface area) and
where
the average pore diameter is in Angstroms, the specific pore volume is in mL/g and the specific surface area is in m²/g.

2. The polymer of claim 1, wherein the polymer has a specific pore volume between 0.5 mL/g and 1.8 mL/g.

3. The polymer of claim 2, wherein the polymer has a specific surface area between 100 m²/g and 700 m²/g.

4. The polymer of claims 1-3, wherein the polymer is a polystyrene/divinylbenzene copolymer.

5. The polymer of claims 1-4, wherein the polymer is adapted to recover monatin from the mixture such that the recovered monatin has a purity level greater than 90%.

6. A method of recovering monatin from a mixture, comprising using a polymer made in the presence of a solvent system, and wherein the solvent system is selected such that it has a dispersion solubility parameter between 15.9 and 18.3 MPa^{1/2}, a polar solubility parameter between 4.0 and 6.2 MPa^{1/2} and a hydrogen bonding solubility parameter between 5.5 and 12.7 MPa^{1/2}.

7. The method of claim 6, wherein the solvent system is selected such that the polymer and the solvent system have a Skaarup distance (Ra) between the polymer and the solvent system of from 7.7 MPa^{1/2} to 10.9 MPa^{1/2}.

8. The method of claims 6-7, wherein the polymer has an average pore diameter between 50 Angstroms to 450 Angstroms, where the average pore diameter is calculated as
Average Pore Diameter=40,000*(specific pore volume)/(specific surface area) and
where
the average pore diameter is in Angstroms, the specific pore volume is in mL/g and the specific surface area is in m²/g.

9. The method of claim 8, wherein the polymer has a specific pore volume between 0.5 mL/g and 1.8 mL/g.

10. The method of claims 8-9, wherein the polymer has a specific surface area between 100 m²/g and 700 m²/g.

11. The method according to claims 6-10, wherein the polymer is a polystyrene/divinylbenzene copolymer.

12. The method according to claims 6-10, wherein the solvent system comprises a solvent selected from the group consisting of chloroform, benzyl alcohol, 1-pentanol, ethyl acetate, toluene, 1-decanol, methyl isobutyl ketone or combinations thereof.

13. The method of claim 6-12, wherein the mixture comprises monatin, monatin precursor and I3P.

14. The method of claim 13, wherein the polymer has a resolution of greater than 0.7 between monatin and monatin precursor.

15. The method of claims 6-14, wherein the polymer is adapted to recover monatin from the mixture such that the recovered monatin has a purity level greater than 90%.
